(19) United States
(12) Patent Application Publication
(10) Pub. No.: US 2025/0143245 A1
(43) Pub. Date: May 8, 2025

(54) COMPOSITIONS AND METHODS RELATED TO SOMATIC EMBRYOGENESIS IN ROSE PLANTS

(71) Applicant: North Carolina State University, Raleigh, NC (US)

(72) Inventors: Kedong Da, Raleigh, NC (US); Thomas G. Ranney, Raleigh, NC (US); Wusheng Liu, Raleigh, NC (US)

(21) Appl. No.: 18/938,794

(22) Filed: Nov. 6, 2024

Related U.S. Application Data
(60) Provisional application No. 63/596,522, filed on Nov. 6, 2023.

(51) Int. Cl. A01H 4/00

(52) U.S. Cl. CPC A01H 4/008; A01H 4/002

(57) ABSTRACT

The present disclosure provides compositions and methods for producing a rose plant using somatic embryogenesis. In particular, the present disclosure provides novel methods and media for rose callus induction, embryoid suspension, and embryo regeneration. Unlike currently available techniques, the novel compositions and methods provided herein produce high yields of competent cells and high frequencies of rose somatic embryogenesis.

### CROSS REFERENCE TO RELATED APPLICATIONS

This application claims priority to and the benefit of U.S. Provisional Patent Application No. 63/596,522 filed Nov. 6, 2023, which is incorporated herein by reference in its entirety for all purposes.

### GOVERNMENT SUPPORT

This invention was made with government support under grant number 58-8020-9-003 awarded by the Agricultural Research Service. The government has certain rights in the invention.

### FIELD

The present disclosure provides compositions and methods for producing a rose plant using somatic embryogenesis. In particular, the present disclosure provides novel methods and media for rose callus induction, embryoid suspension, and embryo regeneration. Unlike currently available techniques, the novel compositions and methods provided herein produce high yields of competent cells and high frequencies of rose somatic embryogenesis.

### BACKGROUND

When referring to cell suspension, the idea of achieving instant synchronous individual cells, and single-cell originated somatic embryos is often discussed. The potential use of cell suspension culture, however, is far beyond practical plant propagation. Cell culture systems are widely used in plant transformation/genome editing, and in many areas of developmental biology. Cell lines may be used to create unique germplasm, for in vitro screening of plants possessing traits such as pathogen and pest resistances, improved quality and stress tolerance produced either via conventional breeding or transgenic means. Cell lines may also be used to produce plant secondary metabolite for industry and medical use. Although various approaches including in vitro organogenesis from plant tissue and organs have been used to produce rose transgenic plants. The only potentially efficient approach is cell somatic embryogenesis, production of transgenic plants through cell cultures. In cell suspension cultures, calli are first induced from various explants, the explants are usually injured by mechanical methods, for example, blade cut or pin holes. Among various auxins used for callus induction, 2,4-Dichlorophenoxyacetic acid (2,4-D) is typically used in most plants. Following callus induction, induced calli are usually suspended in induction medium similar to those in callus induction. Embryo like structure may appear 6-8 month after culture initiation. Factors affecting successful cell cultures include growth condition, cell density, concentration and nature of ingredients used in induction and plant regeneration culture, and physical conditions during the culture process. Generally, low yield of competent cells (especially for elite germplasm), difficulty in plant regeneration and low frequency of somatic embryo (SE) germination are major limiting factors that hindered the use of rose cell culture technology for practical breeding.

### SUMMARY

Embodiments of the present disclosure include methods for producing a rose plant using somatic embryogenesis. In accordance with these embodiments, the methods include culturing at least one rose plantlet leaf in callus induction medium (CIM); isolating at least one callus from the at least one rose plantlet leaf and transferring the at least one callus into rose cell suspension culture media (RSM): isolating at least one embryoid produced from the at least one callus and transferring the at least one embryoid into a somatic embryo regeneration medium (ERM-I); and isolating at least one shoot produced from the at least one embryoid and transferring the at least one shoot to root induction media (RIM). In some embodiments, the at least one shoot develops into a regenerated rose plant.

In some embodiments, the embryoid has insufficient root development and/or embryo clustering. In some embodiments, the method further comprises isolating an embryoid produced from the callus and transferring the embryoid into a somatic embryo regeneration medium II (ERM-II).

In some embodiments, the at least one rose plantlet leaf is from an in vitro plantlet. In some embodiments, the at least one rose plantlet leaf is cultured with its abaxial side in contact with the CIM. In some embodiments, the at least one callus is collected about 4 weeks after being cultured in the CIM.

In some embodiments, the at least one callus is transferred into a suspension culture. In some embodiments, the at least one callus is cultured in RSM at a temperature ranging from about 26° C. to about 30° C. In some embodiments, the RSM is refreshed about every 7 days after culture initiation. In some embodiments, the at least one callus is cultured in RSM for at least about 4 months, or until the at least one callus becomes an embryoid that is at least 2 mm in diameter.

In some embodiments, the at least one embryoid is cultured in ERM-I at a temperature ranging from about 23° C. to about 27° C. In some embodiments, the at least one embryoid is cultured in ERM-I for at least about 4 weeks, or until the at least one embryoid becomes a shoot that is at least 2 cm in height.

Embodiments of the present disclosure include a callus induction medium (CIM) comprising at least one macro salt: at least one iron source: at least one micro salt: at least one vitamin; and at least one growth regulator. In some embodiments, culturing at least one rose plantlet leaf in CIM is sufficient to produce at least one callus.

In some embodiments, the at least one macro salt is present at a concentration ranging from about 50 mg/L to about 1000 mg/L. In some embodiments, the at least one macro salt is selected from the group consisting of NH₄NO₃, KNO₃, CaCl₂). KH₂PO₄, and MgSO₄. In some embodiments, the at least one iron source is present at a concentration ranging from about 25 mg/L to about 40 mg/L. In some embodiments, the at least one iron source is selected from the group consisting of FeSO₄ and Na₂EDTA. In some embodiments, the at least one micro salt is present at a concentration ranging from about 0.2 mg/L to about 25 mg/L. In some embodiments, the at least one micro salt is selected from the group consisting of H₃BO₃, KI. MnSO₄, ZnSO₄, CoCl₂, CuSO₄, and Na₂MoO₄. In some embodiments, the at least one vitamin is present at a concentration ranging from about 0.1 mg/L to about 100 mg/L. In some embodiments, the at least one vitamin is selected from the group consisting of Myo-Inositol. Nicotinic acid. Pyridoxine. Thiamine HCl. Glycine, and L-Proline. In some embodiments, the at least one growth regulator is present at a concentration ranging from about 1.0 mg/L to about 5.0 mg/L. In some embodiments, the at least one growth regulator is selected from the group consisting of 2,4-Dichlorophenoxyacetic acid (2,4-D) and 1-Naphthaleneacetic acid (NAA). In some embodiments, the media further comprises at least one of sucrose and/or gelrite. In some embodiments, the media comprises a pH ranging from about 5.0 to about 6.5.

Embodiments of the present disclosure also include a rose cell suspension culture media (RSM) comprising at least one macro salt: at least one iron source: at least one micro salt: at least one vitamin; and at least one growth regulator. In some embodiments, culturing at least one rose callus in RSM is sufficient to produce at least one embryoid.

In some embodiments, the at least one macro salt is present at a concentration ranging from about 150 mg/L to about 2500 mg/L. In some embodiments, the at least one macro salt is selected from the group consisting of NH₄NO₃, KNO₃, CaCl₂). KH₂PO₄, and MgSO₄. In some embodiments, the at least one iron source is present at a concentration ranging from about 25 mg/L to about 40 mg/L. In some embodiments, the at least one iron source is selected from the group consisting of FeSO₄ and Na₂EDTA. In some embodiments, the at least one micro salt is present at a concentration ranging from about 0.5 mg/L to about 5.0 mg/L. In some embodiments, the at least one micro salt is selected from the group consisting of H₃BO₃, KI, MnSO₄, and ZnSO₄. In some embodiments, the at least one vitamin is present at a concentration ranging from about 0.1 mg/L to about 250 mg/L. In some embodiments, the at least one vitamin is selected from the group consisting of Nicotinic acid, Thiamine HCl, Glycine, L-Proline, Asparagine, and Glutamine. In some embodiments, the at least one growth regulator is present at a concentration ranging from about 0.1 mg/L to about 1.5 mg/L. In some embodiments, the at least one growth regulator is selected from the group consisting of Kinetin (KT) and 2,4-Dichlorophenoxyacetic acid (2,4-D). In some embodiments, the media further comprises at least one of sucrose and/or maltose. In some embodiments, the media comprises a pH ranging from about 5.0 to about 6.5.

Embodiments of the present disclosure also include a rose somatic embryo regeneration medium (ERM-I) comprising at least one macro salt: at least one iron source: at least one micro salt: at least one vitamin; and at least one growth regulator. In some embodiments, culturing at least one rose embryoid in RSM is sufficient to produce at least one shoot.

In some embodiments, the at least one macro salt is present at a concentration ranging from about 150) mg/L to about 2000 mg/L. In some embodiments, the at least one macro salt is selected from the group consisting of NH₄NO₃, KNO₃, CaCl₂, KH₂PO₄, and MgSO₄. In some embodiments, the at least one iron source is present at a concentration ranging from about 35 mg/L to about 60 mg/L. In some embodiments, the at least one iron source is selected from the group consisting of FeSO₄ and Na₂EDTA. In some embodiments, the at least one micro salt is present at a concentration ranging from about 0.02 mg/L to about 25.0 mg/L. In some embodiments, the at least one micro salt is selected from the group consisting of H₃BO₃, KI, MnSO₄, ZnSO₄, CoCl₂, CuSO₄, and Na₂MoO₄. In some embodiments, the at least one vitamin is present at a concentration ranging from about 0.01 mg/L to about 150 mg/L. In some embodiments, the at least one vitamin is selected from the group consisting of Myo-Inositol, Nicotinic acid, Pyridoxine, Thiamine HCl, Glycine, L-Proline, Asparagine, Glutamine, and Boitin. In some embodiments, the at least one growth regulator is present at a concentration ranging from about 0.1 mg/L to about 1.0 mg/L. In some embodiments, the at least one growth regulator is 2,4-D. In some embodiments, the media further comprises at least one of sucrose and/or agar. In some embodiments, the media comprises a pH ranging from about 5.0 to about 6.5.

Embodiments of the present disclosure also include a rose root induction media (RIM) comprising at least one macro salt: at least one iron source: at least one micro salt: at least one vitamin; and at least one growth regulator. In some embodiments, culturing at least one rose shoot in RIM is sufficient to produce at least one regenerated rose plant.

In some embodiments, the at least one macro salt is present at a concentration ranging from about 150 mg/L to about 2000 mg/L. In some embodiments, the at least one macro salt is selected from the group consisting of NH₄NO₃. KNO₃, CaCl₂, KH₂PO₄, and MgSO₄. In some embodiments, the at least one iron source is present at a concentration ranging from about 25 mg/L to about 250 mg/L. In some embodiments, the at least one iron source is selected from the group consisting of FeSO₄. Na₂EDTA, and Sequestrene. In some embodiments, the at least one micro salt is present at a concentration ranging from about 0.02 mg/L to about 25.0 mg/L. In some embodiments, the at least one micro salt is selected from the group consisting of HaBO₃, KI, MnSO₄, ZnSO₄, CoCl₂, CuSO₄, and Na₂MoO₄. In some embodiments, the at least one vitamin is present at a concentration ranging from about 0.05 mg/L to about 150 mg/L. In some embodiments, the at least one vitamin is selected from the group consisting of Myo-Inositol. Nicotinic acid. Pyridoxine. Thiamine HCl. Glycine, and L-Proline. In some embodiments, the at least one growth regulator is present at a concentration ranging from about 0.1 mg/L to about 2.5 mg/L. In some embodiments, the at least one growth regulator is selected from the group consisting of Indole-3-butyric acid (IBA) and Indole-3-acetic acid (IAA). In some embodiments, the media further comprises at least one of sucrose and/or agar. In some embodiments, the media comprises a pH ranging from about 5.0 to about 6.5.

Embodiments of the present disclosure include a rose somatic embryo regeneration medium II (ERM-II) comprising at least one macro salt: at least one iron source: at least one micro salt: at least one vitamin; and at least one growth regulator. In some embodiments, culturing at least one rose embryoid in ERM-II is sufficient to produce at least one shoot.

In some embodiments, the at least one macro salt is present at a concentration ranging from about 150 mg/L to about 2000 mg/L. In some embodiments, the at least one macro salt is selected from the group consisting of NH₄NO₃, KNO₃, CaCl₂, KH₂PO₄, and MgSO₄. In some embodiments, the at least one iron source is present at a concentration ranging from about 25 mg/L to about 40 mg/L. In some embodiments, the at least one iron source is selected from the group consisting of FeSO₄ and Na₂EDTA. In some embodiments, the at least one micro salt is present at a concentration ranging from about 0.02 mg/L to about 25.0 mg/L. In some embodiments, the at least one micro salt is selected from the group consisting of H₃BO₃, KI, MnSO₄, ZnSO₄, CoCl₂, CuSO₄, and Na₂MoO₄. In some embodiments, the at least one vitamin is present at a concentration ranging from about 0.05 mg/L to about 150 mg/L. In some embodiments, the at least one vitamin is selected from the group consisting of Myo-Inositol, Nicotinic acid, Pyridoxine, Thiamine HCl, Glycine, and L-Proline. In some embodiments, the at least one growth regulator is present at a concentration ranging from about 0.1 mg/L to about 2.5 mg/L. In some embodiments, the at least one growth regulator is selected from the group consisting of 6-Benzylaminopurine (BA) and 1-Naphthaleneacetic acid (NAA). In some embodiments, the media further comprises at least one of sucrose and/or agar. In some embodiments, the media comprises a pH ranging from about 5.0 to about 6.5.

### BRIEF DESCRIPTION OF THE DRAWINGS

FIGS. 1A-IF: Representative images of the various stages of rose regeneration through somatic embryogenesis, including: (A) leaf callus induction for 4 weeks: (B) cell suspension culture for 6 months: (C) embryogenic callus in regeneration media: (D) somatic embryogenesis: (E) primary and secondary somatic embryos; and (F) somatic embryos ready for germination/growth.

FIGS. 2A-2D: Representative images of the various stages of rose plant propagation, including: (A) root induction for 4 weeks: (B) well-developed plantlets in rooting media: (C) plantlet acclimatization in a 32-cell tray; and (D) flowering of cell culture regenerated plants.

FIG. 3: Representative flow chart illustrating the various phases of rose cell suspension, including micropropagation through shoot tip culture: cell suspension culture and regeneration; and acclimatization.

### DETAILED DESCRIPTION

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document (including definitions) will control, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a.” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

As used herein, “auxins” generally refer to plant growth hormones that promote cell division and growth.

As used herein, “cytokinins” generally refer to plant growth hormones that affect the organization of dividing cells.

As used herein, “callus” is generally considered to be a growth of unorganized and either unconnected or loosely connected plant cells generally produced from culturing an explant.

As used herein, “embryogenic callus” or “embryoid” generally refer to a translucent white mucilaginous mass that contains early stage embryos attached to suspensors. This is also referred to as an “embryonal-suspensor mass” or “ESM.”

As used herein, “shoot” generally refers to any plant stem together with its appendages, leaves and lateral buds, flowering stems, and flower buds.

As used herein, “explant” generally refers to a piece of tissue taken from a donor plant for culturing.

As used herein, “plantlet” generally refers to a plant asexually reproduced by tissue culture.

As used herein “somatic embryogenesis” generally refers to a process of using tissue culture techniques for generating multiple embryos from an explant. The embryos generated from a given tissue source are believed to be genetically identical.

### 2. COMPOSITIONS AND METHODS

Embodiments of the present disclosure include methods for producing a rose plant using somatic embryogenesis. In accordance with these embodiments, the methods include culturing at least one rose plantlet leaf in callus induction medium (CIM): isolating at least one callus from the at least one rose plantlet leaf and transferring the at least one callus into rose cell suspension culture media (RSM): isolating at least one embryoid produced from the at least one callus and transferring the at least one embryoid into a somatic embryo regeneration medium (ERM-I); and isolating at least one shoot produced from the at least one embryoid and transferring the at least one shoot to root induction media (RIM). In some embodiments, the at least one shoot develops into a regenerated rose plant.

In some embodiments, the embryoid has insufficient root development and/or embryo clustering. In some embodiments, the method further comprises isolating an embryoid produced from the callus and transferring the embryoid into a somatic embryo regeneration medium II (ERM-II).

In some embodiments, the at least one rose plantlet leaf is an in vitro plantlet. In some embodiments, the at least one rose plantlet leaf is cultured with its abaxial side in contact with the CIM. In some embodiments, the at least one callus is collected about 4 weeks after being cultured in the CIM.

In some embodiments, the at least one callus is transferred into a suspension culture. In some embodiments, the at least one callus is cultured in RSM at a temperature ranging from about 26° C. to about 30° C. In some embodiments, the at least one callus is cultured in RSM at a temperature ranging from about 27° C. to about 29° C. In some embodiments, the at least one callus is cultured in RSM at a temperature ranging from about 26° C. to about 28° C. In some embodiments, the at least one callus is cultured in RSM at a temperature ranging from about 28° C. to about 30° C.

In some embodiments, the RSM is refreshed about every 5 to 10 days after culture initiation. In some embodiments, the RSM is refreshed about every 6 to 9 days after culture initiation. In some embodiments, the RSM is refreshed about every 7 to 8 days after culture initiation. In some embodiments, the RSM is refreshed about every 5 to 8 days after culture initiation. In some embodiments, the RSM is refreshed about every 8 to 10 days after culture initiation. In some embodiments, the RSM is refreshed about every 5 days after culture initiation. In some embodiments, the RSM is refreshed about every 6 days after culture initiation. In some embodiments, the RSM is refreshed about every 7 days after culture initiation. In some embodiments, the RSM is refreshed about every 8 days after culture initiation. In some embodiments, the RSM is refreshed about every 9 days after culture initiation. In some embodiments, the RSM is refreshed about every 10 days after culture initiation.

In some embodiments, the at least one callus is cultured in RSM for at least about 3 to 5 months, or until the at least one callus becomes an embryoid that is at least 2 mm in diameter. In some embodiments, the at least one callus is cultured in RSM for at least about 4 to 5 months, or until the at least one callus becomes an embryoid that is at least 2 mm in diameter. In some embodiments, the at least one callus is cultured in RSM for at least about 3 to 4 months, or until the at least one callus becomes an embryoid that is at least 2 mm in diameter. In some embodiments, the at least one callus is cultured in RSM for at least about 3 months. In some embodiments, the at least one callus is cultured in RSM for at least about 4 months. In some embodiments, the at least one callus is cultured in RSM for at least about 5 months.

In some embodiments, the at least one embryoid is cultured in ERM-I at a temperature ranging from about 23° C. to about 27° C. In some embodiments, the at least one embryoid is cultured in ERM-I at a temperature ranging from about 24° C. to about 26° C. In some embodiments, the at least one embryoid is cultured in ERM-I at a temperature ranging from about 23° C. to about 26° C. In some embodiments, the at least one embryoid is cultured in ERM-I at a temperature ranging from about 25° C. to about 27° C.

In some embodiments, the at least one embryoid is cultured in ERM-I for at least about 3 to 5 weeks, or until the at least one embryoid becomes a shoot that is at least 2 cm in height. In some embodiments, the at least one embryoid is cultured in ERM-I for at least about 4 to 5 weeks, or until the at least one embryoid becomes a shoot that is at least 2 cm in height. In some embodiments, the at least one embryoid is cultured in ERM-I for at least about 3 weeks. In some embodiments, the at least one embryoid is cultured in ERM-I for at least about 4 weeks. In some embodiments, the at least one embryoid is cultured in ERM-I for at least about 5 weeks.

Embodiments of the present disclosure include a callus induction medium (CIM) comprising at least one macro salt: at least one iron source: at least one micro salt: at least one vitamin; and at least one growth regulator. In some embodiments, culturing at least one rose plantlet leaf in CIM is sufficient to produce at least one callus.

In some embodiments, the at least one macro salt is present at a concentration ranging from about 50 mg/L to about 1000 mg/L. In some embodiments, the at least one macro salt is selected from the group consisting of NH₄NO₃, KNO₃, CaCl₂), KH₂PO₄, and MgSO₄. In some embodiments, the at least one macro salt is present at a concentration ranging from about 250 mg/L to about 1000 mg/L. In some embodiments, the at least one macro salt is present at a concentration ranging from about 500 mg/L to about 1000 mg/L. In some embodiments, the at least one macro salt is present at a concentration ranging from about 750 mg/L to about 1000 mg/L. In some embodiments, the at least one macro salt is present at a concentration ranging from about 50 mg/L to about 750 mg/L. In some embodiments, the at least one macro salt is present at a concentration ranging from about 50 mg/L to about 500 mg/L. In some embodiments, the at least one macro salt is present at a concentration ranging from about 250 mg/L to about 1000 mg/L.

In some embodiments, the at least one iron source is selected from the group consisting of FeSO₄ and Na₂EDTA. In some embodiments, the at least one iron source is present at a concentration ranging from about 25 mg/L to about 40 mg/L. In some embodiments, the at least one iron source is present at a concentration ranging from about 25 mg/L to about 35 mg/L. In some embodiments, the at least one iron source is present at a concentration ranging from about 25 mg/L to about 30 mg/L. In some embodiments, the at least one iron source is present at a concentration ranging from about 30 mg/L to about 40 mg/L. In some embodiments, the at least one iron source is present at a concentration ranging from about 35 mg/L to about 40 mg/L.

In some embodiments, the at least one micro salt is selected from the group consisting of H₃BO₃, KI, MnSO₄, ZnSO₄, CoCl₂, CuSO₄, and Na₂MoO₄. In some embodiments, the at least one micro salt is present at a concentration ranging from about 0.2 mg/L to about 25 mg/L. In some embodiments, the at least one micro salt is present at a concentration ranging from about 1.0 mg/L to about 25 mg/L. In some embodiments, the at least one micro salt is present at a concentration ranging from about 5.0 mg/L to about 25 mg/L. In some embodiments, the at least one micro salt is present at a concentration ranging from about 10 mg/L to about 25 mg/L. In some embodiments, the at least one micro salt is present at a concentration ranging from about 15 mg/L to about 25 mg/L. In some embodiments, the at least one micro salt is present at a concentration ranging from about 20 mg/L to about 25 mg/L. In some embodiments, the at least one micro salt is present at a concentration ranging from about 0.2 mg/L to about 20 mg/L. In some embodiments, the at least one micro salt is present at a concentration ranging from about 0.2 mg/L to about 15 mg/L. In some embodiments, the at least one micro salt is present at a concentration ranging from about 0.2 mg/L to about 10 mg/L. In some embodiments, the at least one micro salt is present at a concentration ranging from about 0.2 mg/L to about 5 mg/L.

In some embodiments, the at least one vitamin is selected from the group consisting of Myo-Inositol. Nicotinic acid. Pyridoxine. Thiamine HCl. Glycine, and L-Proline. In some embodiments, the at least one vitamin is present at a concentration ranging from about 0.1 mg/L to about 100 mg/L. In some embodiments, the at least one vitamin is present at a concentration ranging from about 1.0 mg/L to about 100 mg/L. In some embodiments, the at least one vitamin is present at a concentration ranging from about 10 mg/L to about 100 mg/L. In some embodiments, the at least one vitamin is present at a concentration ranging from about 25 mg/L to about 100 mg/L. In some embodiments, the at least one vitamin is present at a concentration ranging from about 50 mg/L to about 100 mg/L. In some embodiments, the at least one vitamin is present at a concentration ranging from about 75 mg/L to about 100 mg/L.

In some embodiments, the at least one growth regulator is selected from the group consisting of 2,4-Dichlorophenoxyacetic acid (2,4-D) and 1-Naphthaleneacetic acid (NAA). In some embodiments, the at least one growth regulator is present at a concentration ranging from about 1.0 mg/L to about 5.0 mg/L. In some embodiments, the at least one growth regulator is present at a concentration ranging from about 2.0 mg/L to about 5.0 mg/L. In some embodiments, the at least one growth regulator is present at a concentration ranging from about 3.0 mg/L to about 5.0 mg/L. In some embodiments, the at least one growth regulator is present at a concentration ranging from about 4.0 mg/L to about 5.0 mg/L. In some embodiments, the at least one growth regulator is present at a concentration ranging from about 1.0 mg/L to about 4.0 mg/L. In some embodiments, the at least one growth regulator is present at a concentration ranging from about 1.0 mg/L to about 3.0 mg/L. In some embodiments, the at least one growth regulator is present at a concentration ranging from about 1.0 mg/L to about 2.0 mg/L.

In some embodiments, the media further comprises at least one of sucrose and/or gelrite. In some embodiments, the media comprises a pH ranging from about 5.0 to about 6.5. In some embodiments, the media comprises a pH ranging from about 5.0 to about 6.0. In some embodiments, the media comprises a pH ranging from about 5.5 to about 6.5.

Embodiments of the present disclosure also include a rose cell suspension culture media (RSM) comprising at least one macro salt: at least one iron source: at least one micro salt: at least one vitamin; and at least one growth regulator. In some embodiments, culturing at least one rose callus in RSM is sufficient to produce at least one embryoid.

In some embodiments, the at least one macro salt is selected from the group consisting of NH₄NO₃, KNO₃, CaCl₂, KH₂PO₄, and MgSO₄. In some embodiments, the at least one macro salt is present at a concentration ranging from about 150 mg/L to about 2500 mg/L. In some embodiments, the at least one macro salt is present at a concentration ranging from about 150 mg/L to about 2000 mg/L. In some embodiments, the at least one macro salt is present at a concentration ranging from about 150 mg/L to about 1500 mg/L. In some embodiments, the at least one macro salt is present at a concentration ranging from about 150 mg/L to about 1000 mg/L. In some embodiments, the at least one macro salt is present at a concentration ranging from about 150 mg/L to about 500 mg/L. In some embodiments, the at least one macro salt is present at a concentration ranging from about 500 mg/L to about 2500 mg/L. In some embodiments, the at least one macro salt is present at a concentration ranging from about 1000 mg/L to about 2500 mg/L. In some embodiments, the at least one macro salt is present at a concentration ranging from about 1500 mg/L to about 2500 mg/L. In some embodiments, the at least one macro salt is present at a concentration ranging from about 2000 mg/L to about 2500 mg/L.

In some embodiments, the at least one iron source is selected from the group consisting of FeSO₄ and Na₂EDTA. In some embodiments, the at least one iron source is present at a concentration ranging from about 25 mg/L to about 40 mg/L. In some embodiments, the at least one iron source is present at a concentration ranging from about 25 mg/L to about 35 mg/L. In some embodiments, the at least one iron source is present at a concentration ranging from about 25 mg/L to about 30 mg/L. In some embodiments, the at least one iron source is present at a concentration ranging from about 30 mg/L to about 40 mg/L. In some embodiments, the at least one iron source is present at a concentration ranging from about 35 mg/L to about 40 mg/L.

In some embodiments, the at least one micro salt is selected from the group consisting of H₃BO₃, KI, MnSO₄, and ZnSO₄. In some embodiments, the at least one micro salt is present at a concentration ranging from about 0.5 mg/L to about 5.0 mg/L. In some embodiments, the at least one micro salt is present at a concentration ranging from about 0.5 mg/L to about 2.5 mg/L. In some embodiments, the at least one micro salt is present at a concentration ranging from about 0.5 mg/L to about 1.0 mg/L. In some embodiments, the at least one micro salt is present at a concentration ranging from about 1.0 mg/L to about 5.0 mg/L. In some embodiments, the at least one micro salt is present at a concentration ranging from about 2.5 mg/L to about 5.0 mg/L.

In some embodiments, the at least one vitamin is selected from the group consisting of Nicotinic acid, Thiamine HCl, Glycine, L-Proline, Asparagine, and Glutamine. In some embodiments, the at least one vitamin is present at a concentration ranging from about 0.1 mg/L to about 250 mg/L. In some embodiments, the at least one vitamin is present at a concentration ranging from about 0.1 mg/L to about 150 mg/L. In some embodiments, the at least one vitamin is present at a concentration ranging from about 0.1 mg/L to about 100 mg/L. In some embodiments, the at least one vitamin is present at a concentration ranging from about 0.1 mg/L to about 50 mg/L. In some embodiments, the at least one vitamin is present at a concentration ranging from about 0.1 mg/L to about 10 mg/L. In some embodiments, the at least one vitamin is present at a concentration ranging from about 10 mg/L to about 250 mg/L. In some embodiments, the at least one vitamin is present at a concentration ranging from about 50 mg/L to about 250 mg/L. In some embodiments, the at least one vitamin is present at a concentration ranging from about 100 mg/L to about 250 mg/L. In some embodiments, the at least one vitamin is present at a concentration ranging from about 150 mg/L to about 250 mg/L.

In some embodiments, the at least one growth regulator is selected from the group consisting of Kinetin (KT) and 2,4-Dichlorophenoxyacetic acid (2,4-D). In some embodiments, the at least one growth regulator is present at a concentration ranging from about 0.1 mg/L to about 1.5 mg/L. In some embodiments, the at least one growth regulator is present at a concentration ranging from about 0.5 mg/L to about 1.5 mg/L. In some embodiments, the at least one growth regulator is present at a concentration ranging from about 1.0 mg/L to about 1.5 mg/L. In some embodiments, the at least one growth regulator is present at a concentration ranging from about 0.1 mg/L to about 1.0 mg/L. In some embodiments, the at least one growth regulator is present at a concentration ranging from about 0.1 mg/L to about 0.5 mg/L.

In some embodiments, the media further comprises at least one of sucrose and/or maltose. In some embodiments, the media comprises a pH ranging from about 5.0 to about 6.5. In some embodiments, the media comprises a pH ranging from about 5.0 to about 6.0. In some embodiments, the media comprises a pH ranging from about 5.5 to about 6.5.

Embodiments of the present disclosure also include a rose somatic embryo regeneration medium (ERM-I) comprising at least one macro salt: at least one iron source: at least one micro salt: at least one vitamin; and at least one growth regulator. In some embodiments, culturing at least one rose embryoid in ERM-I is sufficient to produce at least one shoot.

In some embodiments, the at least one macro salt is selected from the group consisting of NH₄NO₃, KNO₃, CaCl₂, KH₂PO₄, and MgSO₄. In some embodiments, the at least one macro salt is present at a concentration ranging from about 150 mg/L to about 2000 mg/L. In some embodiments, the at least one macro salt is present at a concentration ranging from about 150 mg/L to about 1500 mg/L. In some embodiments, the at least one macro salt is present at a concentration ranging from about 150 mg/L to about 1000 mg/L. In some embodiments, the at least one macro salt is present at a concentration ranging from about 150 mg/L to about 500 mg/L. In some embodiments, the at least one macro salt is present at a concentration ranging from about 500 mg/L to about 2000 mg/L. In some embodiments, the at least one macro salt is present at a concentration ranging from about 150 mg/L to about 1000 mg/L. In some embodiments, the at least one macro salt is present at a concentration ranging from about 150 mg/L to about 1500 mg/L.

In some embodiments, the at least one iron source is selected from the group consisting of FeSO₄ and Na₂EDTA. In some embodiments, the at least one iron source is present at a concentration ranging from about 35 mg/L to about 60 mg/L. In some embodiments, the at least one iron source is present at a concentration ranging from about 35 mg/L to about 50 mg/L. In some embodiments, the at least one iron source is present at a concentration ranging from about 35 mg/L to about 40 mg/L. In some embodiments, the at least one iron source is present at a concentration ranging from about 40 mg/L to about 60 mg/L. In some embodiments, the at least one iron source is present at a concentration ranging from about 50 mg/L to about 60 mg/L.

In some embodiments, the at least one micro salt is selected from the group consisting of H₃BO₃, KI, MnSO₄, ZnSO₄, CoCl₂, CuSO₄, and Na₂MoO₄. In some embodiments, the at least one micro salt is present at a concentration ranging from about 0.02 mg/L to about 25.0 mg/L. In some embodiments, the at least one micro salt is present at a concentration ranging from about 0.1 mg/L to about 25.0 mg/L. In some embodiments, the at least one micro salt is present at a concentration ranging from about 1.0 mg/L to about 25.0 mg/L. In some embodiments, the at least one micro salt is present at a concentration ranging from about 5.0 mg/L to about 25.0 mg/L. In some embodiments, the at least one micro salt is present at a concentration ranging from about 10 mg/L to about 25.0 mg/L. In some embodiments, the at least one micro salt is present at a concentration ranging from about 15 mg/L to about 25.0 mg/L. In some embodiments, the at least one micro salt is present at a concentration ranging from about 20 mg/L to about 25.0 mg/L. In some embodiments, the at least one micro salt is present at a concentration ranging from about 0.02 mg/L to about 20 mg/L. In some embodiments, the at least one micro salt is present at a concentration ranging from about 0.02 mg/L to about 15 mg/L. In some embodiments, the at least one micro salt is present at a concentration ranging from about 0.02 mg/L to about 10 mg/L. In some embodiments, the at least one micro salt is present at a concentration ranging from about 0.02 mg/L to about 5.0 mg/L. In some embodiments, the at least one micro salt is present at a concentration ranging from about 1.0 mg/L to about 25.0 mg/L.

In some embodiments, the at least one vitamin is selected from the group consisting of Myo-Inositol. Nicotinic acid. Pyridoxine. Thiamine HCl. Glycine. L-Proline. Asparagine. Glutamine, and Boitin. In some embodiments, the at least one vitamin is present at a concentration ranging from about 0.01 mg/L to about 150 mg/L. In some embodiments, the at least one vitamin is present at a concentration ranging from about 0.01 mg/L to about 100 mg/L. In some embodiments, the at least one vitamin is present at a concentration ranging from about 0.01 mg/L to about 50 mg/L. In some embodiments, the at least one vitamin is present at a concentration ranging from about 0.01 mg/L to about 10 mg/L. In some embodiments, the at least one vitamin is present at a concentration ranging from about 1.0 mg/L to about 150 mg/L. In some embodiments, the at least one vitamin is present at a concentration ranging from about 10 mg/L to about 150 mg/L. In some embodiments, the at least one vitamin is present at a concentration ranging from about 50 mg/L to about 150 mg/L. In some embodiments, the at least one vitamin is present at a concentration ranging from about 100 mg/L to about 150 mg/L.

In some embodiments, the at least one growth regulator is 2,4-D. In some embodiments, the at least one growth regulator is present at a concentration ranging from about 0.1 mg/L to about 1.0 mg/L. In some embodiments, the at least one growth regulator is present at a concentration ranging from about 0.1 mg/L to about 0.5 mg/L. In some embodiments, the at least one growth regulator is present at a concentration ranging from about 0.1 mg/L to about 0.25 mg/L. In some embodiments, the at least one growth regulator is present at a concentration ranging from about 0.25 mg/L to about 1.0 mg/L. In some embodiments, the at least one growth regulator is present at a concentration ranging from about 0.5 mg/L to about 1.0 mg/L.

In some embodiments, the media further comprises at least one of sucrose and/or agar. In some embodiments, the media comprises a pH ranging from about 5.0 to about 6.5. In some embodiments, the media comprises a pH ranging from about 5.0 to about 6.0. In some embodiments, the media comprises a pH ranging from about 5.5 to about 6.5.

Embodiments of the present disclosure also include a rose root induction media (RIM) comprising at least one macro salt: at least one iron source: at least one micro salt: at least one vitamin; and at least one growth regulator. In some embodiments, culturing at least one rose shoot in RIM is sufficient to produce at least one regenerated rose plant.

In some embodiments, the at least one macro salt is selected from the group consisting of NH₄NO₃, KNO₃, CaCl₂, KH₂PO₄, and MgSO₄. In some embodiments, the at least one macro salt is present at a concentration ranging from about 150 mg/L to about 2000 mg/L. In some embodiments, the at least one macro salt is present at a concentration ranging from about 150 mg/L to about 1500 mg/L. In some embodiments, the at least one macro salt is present at a concentration ranging from about 150 mg/L to about 1000 mg/L. In some embodiments, the at least one macro salt is present at a concentration ranging from about 150 mg/L to about 500 mg/L. In some embodiments, the at least one macro salt is present at a concentration ranging from about 500 mg/L to about 2000 mg/L. In some embodiments, the at least one macro salt is present at a concentration ranging from about 1000 mg/L to about 2000 mg/L. In some embodiments, the at least one macro salt is present at a concentration ranging from about 1500 mg/L to about 2000 mg/L.

In some embodiments, the at least one iron source is selected from the group consisting of FeSO₄, Na₂EDTA, and Sequestrene. In some embodiments, the at least one iron source is present at a concentration ranging from about 25 mg/L to about 250 mg/L. In some embodiments, the at least one iron source is present at a concentration ranging from about 25 mg/L to about 200 mg/L. In some embodiments, the at least one iron source is present at a concentration ranging from about 25 mg/L to about 150 mg/L. In some embodiments, the at least one iron source is present at a concentration ranging from about 25 mg/L to about 100 mg/L. In some embodiments, the at least one iron source is present at a concentration ranging from about 25 mg/L to about 50 mg/L. In some embodiments, the at least one iron source is present at a concentration ranging from about 50 mg/L to about 250 mg/L. In some embodiments, the at least one iron source is present at a concentration ranging from about 100 mg/L to about 250 mg/L. In some embodiments, the at least one iron source is present at a concentration ranging from about 150 mg/L to about 250 mg/L. In some embodiments, the at least one iron source is present at a concentration ranging from about 200 mg/L to about 250 mg/L.

In some embodiments, the at least one micro salt is selected from the group consisting of H₃BO₃, KI, MnSO₄, ZnSO₄, CoCl₂, CuSO₄, and Na₂MoO₄. In some embodiments, the at least one micro salt is present at a concentration ranging from about 0.02 mg/L to about 25.0 mg/L. In some embodiments, the at least one micro salt is present at a concentration ranging from about 0.02 mg/L to about 20 mg/L. In some embodiments, the at least one micro salt is present at a concentration ranging from about 0.02 mg/L to about 15 mg/L. In some embodiments, the at least one micro salt is present at a concentration ranging from about 0.02 mg/L to about 10 mg/L. In some embodiments, the at least one micro salt is present at a concentration ranging from about 0.02 mg/L to about 5.0 mg/L. In some embodiments, the at least one micro salt is present at a concentration ranging from about 0.02 mg/L to about 1.0 mg/L. In some embodiments, the at least one micro salt is present at a concentration ranging from about 1.0 mg/L to about 25 mg/L. In some embodiments, the at least one micro salt is present at a concentration ranging from about 5.0 mg/L to about 25 mg/L. In some embodiments, the at least one micro salt is present at a concentration ranging from about 10 mg/L to about 25 mg/L. In some embodiments, the at least one micro salt is present at a concentration ranging from about 15 mg/L to about 25 mg/L. In some embodiments, the at least one micro salt is present at a concentration ranging from about 20 mg/L to about 25 mg/L.

In some embodiments, the at least one vitamin is selected from the group consisting of Myo-Inositol. Nicotinic acid. Pyridoxine. Thiamine HCl. Glycine, and L-Proline. In some embodiments, the at least one vitamin is present at a concentration ranging from about 0.05 mg/L to about 150 mg/L. In some embodiments, the at least one vitamin is present at a concentration ranging from about 0.05 mg/L to about 100 mg/L. In some embodiments, the at least one vitamin is present at a concentration ranging from about 0.05 mg/L to about 50 mg/L. In some embodiments, the at least one vitamin is present at a concentration ranging from about 0.05 mg/L to about 25 mg/L. In some embodiments, the at least one vitamin is present at a concentration ranging from about 0.05 mg/L to about 10 mg/L. In some embodiments, the at least one vitamin is present at a concentration ranging from about 0.05 mg/L to about 1.0 mg/L. In some embodiments, the at least one vitamin is present at a concentration ranging from about 1.0 mg/L to about 150 mg/L. In some embodiments, the at least one vitamin is present at a concentration ranging from about 10 mg/L to about 150 mg/L. In some embodiments, the at least one vitamin is present at a concentration ranging from about 25 mg/L to about 150 mg/L. In some embodiments, the at least one vitamin is present at a concentration ranging from about 50 mg/L to about 150 mg/L. In some embodiments, the at least one vitamin is present at a concentration ranging from about 100 mg/L to about 150 mg/L.

In some embodiments, the at least one growth regulator is selected from the group consisting of Indole-3-butyric acid (IBA) and Indole-3-acetic acid (IAA). In some embodiments, the at least one growth regulator is present at a concentration ranging from about 0.1 mg/L to about 2.5 mg/L. In some embodiments, the at least one growth regulator is present at a concentration ranging from about 0.1 mg/L to about 2.0 mg/L. In some embodiments, the at least one growth regulator is present at a concentration ranging from about 0.1 mg/L to about 1.0 mg/L. In some embodiments, the at least one growth regulator is present at a concentration ranging from about 0.1 mg/L to about 0.5 mg/L. In some embodiments, the at least one growth regulator is present at a concentration ranging from about 0.5 mg/L to about 2.5 mg/L. In some embodiments, the at least one growth regulator is present at a concentration ranging from about 1.0 mg/L to about 2.5 mg/L. In some embodiments, the at least one growth regulator is present at a concentration ranging from about 2.0 mg/L to about 2.5 mg/L.

In some embodiments, the media further comprises at least one of sucrose and/or agar. In some embodiments, the media comprises a pH ranging from about 5.0 to about 6.5. In some embodiments, the media comprises a pH ranging from about 5.0 to about 6.0. In some embodiments, the media comprises a pH ranging from about 5.5 to about 6.5.

Embodiments of the present disclosure include a rose somatic embryo regeneration medium II (ERM-II) comprising at least one macro salt: at least one iron source: at least one micro salt: at least one vitamin; and at least one growth regulator. In some embodiments, culturing at least one rose embryoid in ERM-II is sufficient to produce at least one shoot.

In some embodiments, the at least one macro salt is selected from the group consisting of NH₄NO₃, KNO₃, CaCl₂, KH₂PO₄, and MgSO₄. In some embodiments, the at least one macro salt is present at a concentration ranging from about 150 mg/L to about 2000 mg/L. In some embodiments, the at least one macro salt is present at a concentration ranging from about 150 mg/L to about 1500 mg/L. In some embodiments, the at least one macro salt is present at a concentration ranging from about 150 mg/L to about 1000 mg/L. In some embodiments, the at least one macro salt is present at a concentration ranging from about 150 mg/L to about 500 mg/L. In some embodiments, the at least one macro salt is present at a concentration ranging from about 150 mg/L to about 250 mg/L. In some embodiments, the at least one macro salt is present at a concentration ranging from about 250 mg/L to about 2000 mg/L. In some embodiments, the at least one macro salt is present at a concentration ranging from about 500 mg/L to about 2000 mg/L. In some embodiments, the at least one macro salt is present at a concentration ranging from about 1000 mg/L to about 2000 mg/L. In some embodiments, the at least one macro salt is present at a concentration ranging from about 1500 mg/L to about 2000 mg/L.

In some embodiments, the at least one iron source is selected from the group consisting of FeSO₄ and Na₂EDTA. In some embodiments, the at least one iron source is present at a concentration ranging from about 25 mg/L to about 40 mg/L. In some embodiments, the at least one iron source is present at a concentration ranging from about 25 mg/L to about 35 mg/L. In some embodiments, the at least one iron source is present at a concentration ranging from about 25 mg/L to about 30 mg/L. In some embodiments, the at least one iron source is present at a concentration ranging from about 30 mg/L to about 40 mg/L. In some embodiments, the at least one iron source is present at a concentration ranging from about 35 mg/L to about 40 mg/L.

In some embodiments, the at least one micro salt is selected from the group consisting of HaBO₃, KI, MnSO₄, ZnSO₄, CoCl₂, CuSO₄, and Na₂MoO₄. In some embodiments, the at least one micro salt is present at a concentration ranging from about 0.02 mg/L to about 25.0 mg/L. In some embodiments, the at least one micro salt is present at a concentration ranging from about 0.02 mg/L to about 20 mg/L. In some embodiments, the at least one micro salt is present at a concentration ranging from about 0.02 mg/L to about 15 mg/L. In some embodiments, the at least one micro salt is present at a concentration ranging from about 0.02 mg/L to about 10 mg/L. In some embodiments, the at least one micro salt is present at a concentration ranging from about 0.02 mg/L to about 5 mg/L. In some embodiments, the at least one micro salt is present at a concentration ranging from about 0.02 mg/L to about 1.0 mg/L. In some embodiments, the at least one micro salt is present at a concentration ranging from about 0.02 mg/L to about 0.5 mg/L. In some embodiments, the at least one micro salt is present at a concentration ranging from about 0.5 mg/L to about 20 mg/L. In some embodiments, the at least one micro salt is present at a concentration ranging from about 1.0 mg/L to about 20 mg/L. In some embodiments, the at least one micro salt is present at a concentration ranging from about 5 mg/L to about 20 mg/L. In some embodiments, the at least one micro salt is present at a concentration ranging from about 10 mg/L to about 20 mg/L. In some embodiments, the at least one micro salt is present at a concentration ranging from about 15 mg/L to about 20 mg/L.

In some embodiments, the at least one vitamin is selected from the group consisting of Myo-Inositol. Nicotinic acid. Pyridoxine. Thiamine HCl. Glycine, and L-Proline. In some embodiments, the at least one vitamin is present at a concentration ranging from about 0.05 mg/L to about 150 mg/L. In some embodiments, the at least one vitamin is present at a concentration ranging from about 0.05 mg/L to about 100 mg/L. In some embodiments, the at least one vitamin is present at a concentration ranging from about 0.05 mg/L to about 50 mg/L. In some embodiments, the at least one vitamin is present at a concentration ranging from about 0.05 mg/L to about 10 mg/L. In some embodiments, the at least one vitamin is present at a concentration ranging from about 0.05 mg/L to about 1.0 mg/L. In some embodiments, the at least one vitamin is present at a concentration ranging from about 1.0 mg/L to about 150 mg/L. In some embodiments, the at least one vitamin is present at a concentration ranging from about 10 mg/L to about 150) mg/L. In some embodiments, the at least one vitamin is present at a concentration ranging from about 50 mg/L to about 150 mg/L. In some embodiments, the at least one vitamin is present at a concentration ranging from about 100 mg/L to about 150 mg/L.

In some embodiments, the at least one growth regulator is selected from the group consisting of 6-Benzylaminopurine (BA) and 1-Naphthaleneacetic acid (NAA). In some embodiments, the at least one growth regulator is present at a concentration ranging from about 0.1 mg/L to about 2.5 mg/L. In some embodiments, the at least one growth regulator is present at a concentration ranging from about 0.1 mg/L to about 2.0 mg/L. In some embodiments, the at least one growth regulator is present at a concentration ranging from about 0.1 mg/L to about 1.5 mg/L. In some embodiments, the at least one growth regulator is present at a concentration ranging from about 0.1 mg/L to about 1.0 mg/L. In some embodiments, the at least one growth regulator is present at a concentration ranging from about 0.1 mg/L to about 0.5 mg/L. In some embodiments, the at least one growth regulator is present at a concentration ranging from about 0.5 mg/L to about 2.5 mg/L. In some embodiments, the at least one growth regulator is present at a concentration ranging from about 1.0 mg/L to about 2.5 mg/L. In some embodiments, the at least one growth regulator is present at a concentration ranging from about 1.5 mg/L to about 2.5 mg/L. In some embodiments, the at least one growth regulator is present at a concentration ranging from about 2.0 mg/L to about 2.5 mg/L.

In some embodiments, the media further comprises at least one of sucrose and/or agar. In some embodiments, the media comprises a pH ranging from about 5.0 to about 6.5. In some embodiments, the media comprises a pH ranging from about 5.0 to about 6.0. In some embodiments, the media comprises a pH ranging from about 5.5 to about 6.5.

### 3. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Embodiments of the present disclosure describe an efficient system for production of rose somatic embryo (SE) plants from cell suspension culture initiated from leaf callus. From the onset of the project, step by step experiments were performed to addressing the major problems in previous systems, including improving the frequency of competent cell induction and increase SE yields. Experiments were conducted to optimize conditions for rose callus induction, to test medium composition for efficient competent cell suspension culture, and to establish high frequency somatic embryogenesis in rose. By optimizing the induction, culture and regeneration conditions, high yields of competent cells were produced, and a high frequency of somatic embryogenesis was achieved. In some cases, over 90% of regenerated somatic embryos developed into plantlets. 90% of regenerated plantlets rooted, and 98% of plants flowered and maintained characteristics of mother plants. This system was first established using a bush rose Italian Ice′ and has since been found applicable to other types of rose plants.

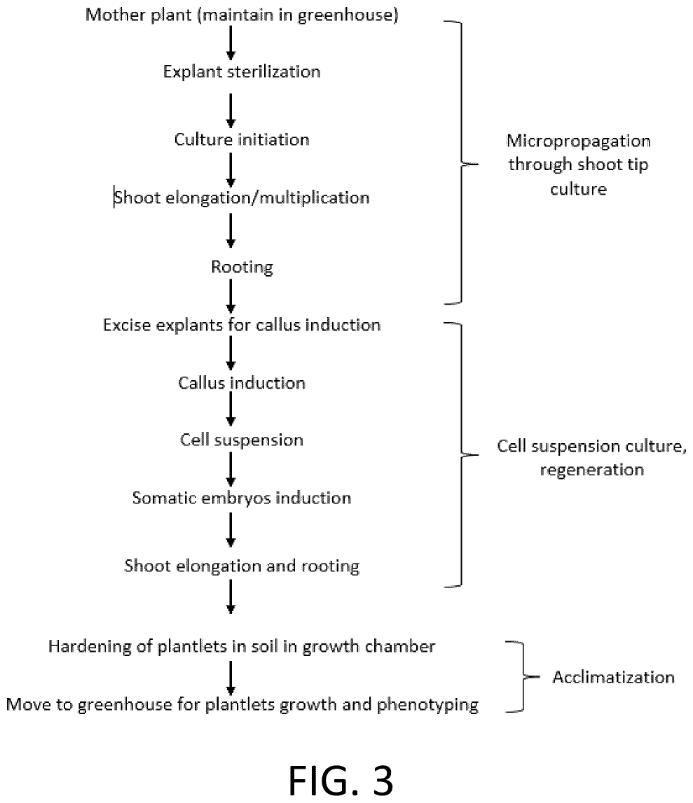

The following example describes one representative embodiment of the compositions and methods of the present disclosure pertaining to an efficient system for production of rose somatic embryo plants from cell suspension culture initiated from leaf callus (FIG. 3). Variations of the compositions and methods described in this example are possible, as would be understood by one of ordinary skill in the art based on the present disclosure.

Donor plant and growth conditions. Mother plants are grown in a gallon pot. The plant may be grown in either a greenhouse or a growth chamber with a day/night temperature regime at 25/20° C., and photoperiod of 16/8 h. In preparation for transplanting, pots filled with pre-mixed soil are first watered to saturation. One plant per pot. Application of fertilizer is achieved by daily watering, which contains 200-250 ppm of water-soluble fertilizer of nitrogen (N), Phosphorus (P₂O₅) and potassium (K₂O), 20% each. In general, any standard conditions for growing rose in a greenhouse or growth chamber are acceptable.

Sampling. For every genotype or variety, label tube name and date correspond with specific genotype or variety. The actively grown meristems about 2 inches are harvested by a pair of pruners. Sampled stems are brought back to the laboratory where all foliage is removed from the stems. The leafless stems are cut into about ½ inch cuttings by a pruner: each cutting contains a node between two cuts. The cuttings are washed under the running tape water for 45 min in the lab.

Sterilization. Prepare the rose elongation medium (REM) plates in accordance with Table 1. Autoclave 500 mL of distilled water at the same time as the elongation medium. Use this later as rinse water. Prepare 100 mL 20% commercial bleach (or 1.2% sodium hypochlorite) solution to be used for sterilizing the cuttings. Add 50 μL of Tween 20 detergent to the bleach solution. In a laminar flow hood, the running water-washed cuttings are drained out of water. Pre-sterilization with 70% ethanol. 15-20 cuttings are blot dried on a piece of sterile paper tower and pre-sterilized in a 50 ml conical centrifuge tube with 25 ml 70% ethanol for 45 seconds. Sterilize the cuttings using the 20% bleach solution. Drain out the 70% ethanol and add 30 ml of 20% bleach solution to the sterile centrifuge tube containing the cuttings. Shake the tube on a rotator platform at 125 rpm to suspend the cuttings in the peroxide solution.

Rinse the bleach solution from the tube. After 20 minutes, remove the bleach solution from the centrifuge tube using an aspirator fitted with a pipette tip on the end. Add 25 mL of sterile distilled water to the tube. Close the tube and shake 2 min. Carefully remove the rinse solution by pipetting. Repeat this rinsing process 3 times.

Plate the sterilized cuttings on germination plates. In a laminar flow hood, label the bottom of the plate with REM medium with the stock name and the current date. Blot-dry the cuttings on a sterile dry paper towel and plant in the REM plate, five cuttings per plate. Place the lid on the germination plate. Seal the REM plate by wrapping the plate with parafilm. Put the plate in a dark growth chamber at the temperature of 25±2° C. for shoot elongation.

Micropropagation. Rose nodes contain dormant buds. The bud elongates on the cutting REM medium after 4 weeks in the dark. Germinated buds produce new white meristems. The new meristems are transferred to a magenta box containing rose micropropagation medium RPM (Table 1) for growth and multiplication/micropropagation in the growth chamber. The growth chamber conditions are set as day/night temperature regime at 25/20° C., and photoperiod of 16/8 h with coolwhite fluorescent light intensity of 115 μmol/m²/s. When the meristem culture increases in size and multiplies, cultures should be sub-cultured on a fresh RPM by separating the plantlets. Micropropagation media are refreshed every 4 weeks. After having targeted the number of plantlets, transfer the plantlets to the rooting media (RIM).

Rooting. Place well-formed shoots on to RIM medium to induce root formation in a 16/8 h light/dark photoperiod using coolwhite fluorescent lights (115 μmol/m²/s) at 25±2° C. The cultures regenerate roots in 6 weeks. Transfer healthy rooted plantlets in soil to a greenhouse or growth chamber. Add water to the plantlets and allow sitting for an hour. This helps loosen the roots from agar. Carefully scoop the plant (roots and agar together) out of the container. Hold the plant upside down under water. Gently wash with tap water to remove residues of agar from the roots. Fill the 32 cell tray half full of sterile substrate mixture. Hold the plant upright in the pot and pour more substrate around the roots until the pot is full of soil and the plant is upright. Water the plant and substrate. Plants that have been brought from the aseptic culture require a hardening-off period to acclimate them to the lower humidity of the growth chamber. In the greenhouse, and especially in the field, irradiance is much higher and air humidity much lower than those in the vessels. Even if the water potential of the substrate is higher than the water potential of the rooting media, the plantlets may quickly wilt, as water loss of their leaves is not restricted. In addition, water supply can be limiting because of low hydraulic conductivity of roots and root-stem connections. Many plantlets die during this period. Therefore, after ex vitro transplantation, plantlets usually need some weeks of acclimatization with a gradual lowering of air humidity.

TABLE 1
Media formulations for rose micropropagation and rooting.

| | REM | RPM-I | RPM-II | RIM |
|---|---|---|---|---|
| Media | medium | medium | medium | medium |
| components | (mg/L) | (mg/L) | (mg/L) | (mg/L) |
| Macro salts | | | | |
| NH₄NO₃ | 50-1000 | 150-2000 | 150-2000 | 150-2000 |
| KNO₃ | 50-1000 | 150-2000 | 150-2000 | 150-2000 |
| CaCl₂ (anhydrous) | 50-1000 | 150-2000 | 150-2000 | 150-2000 |
| KH₂PO₄ | 50-1000 | 150-2000 | 150-2000 | 150-2000 |
| MgSO₄•7H₂O | 50-1000 | 150-2000 | 150-2000 | 150-2000 |
| Iron source | | | | |
| FeSO₄•7H₂O | 25-40 | 35-60 | 35-60 | 25-250 |
| Na₂EDTA | 25-40 | 35-60 | 35-60 | 25-250 |
| Sequestrene | | | | 25-250 |
| Micro salts | | | | |
| H₃BO₃ | 0.2-25 | 0.2-25 | 0.2-25 | 0.2-25 |
| KI | 0.2-25 | 0.2-25 | 0.2-25 | 0.2-25 |
| MnSO₄•4H₂O | 0.2-25 | 0.2-25 | 0.2-25 | 0.2-25 |
| ZnSO₄•7H₂O | 0.2-25 | 0.2-25 | 0.2-25 | 0.2-25 |
| CoCl₂•6H₂O | 0.2-25 | 0.2-25 | 0.2-25 | 0.2-25 |
| CuSO₄•5H₂O | 0.2-25 | 0.2-25 | 0.2-25 | 0.2-25 |
| Na₂MoO4•2H₂O | 0.2-25 | 0.2-25 | 0.2-25 | 0.2-25 |
| Vitamins | | | | |
| Myo-Inositol | 0.5-150 | 0.01-150 | 0.01-150 | 0.5-150 |
| Nicotinic acid | 0.5-150 | 0.01-150 | 0.01-150 | 0.5-150 |
| Pyridoxine | 0.5-150 | 0.01-150 | 0.01-150 | 0.5-150 |
| Thiamine HCl | 0.5-150 | 0.01-150 | 0.01-150 | 0.5-150 |
| Glycine | 0.5-150 | 0.01-150 | 0.01-150 | 0.5-150 |
| L-Proline | 0.5-150 | 0.01-150 | 0.01-150 | 0.5-150 |
| Asparagine | — | 0.01-150 | 0.01-150 | — |
| Glutamine | — | 0.01-150 | 0.01-150 | — |
| Boitin | — | 0.01-150 | 0.01-150 | — |
| Growth regulators | | | | |
| BA | 1.0-5.0 | 1.0-5.0 | 0.1-5.0 | — |
| 2,4-D | — | — | — | — |
| NAA | — | 0.1-5.0 | — | — |
| IBA | — | — | — | 0.1-5.0 |
| IAA | — | — | — | 0.1-5.0 |
| Other components | | | | |
| Sucrose | 10-20 | 25-50 | 25-50 | 25-50 |
| Gelrite | 2-6 | — | — | — |
| Agar | — | 5-10 | 5-10 | 5-10 |

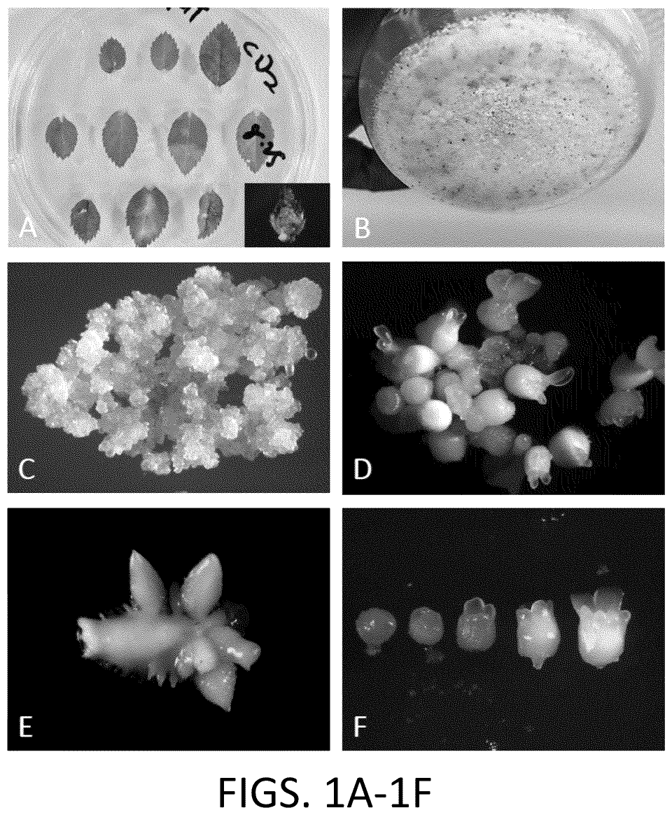

Callus induction. Collect the first three open leaves from an in vitro plantlet for callus induction, culture the leaves on callus induction medium (CIM) (Table 2) with abaxial side touch the medium. First callus appears at the base part of the cultures, and callus is induced in other parts of the leaf with time (FIG. 1A). All calluses are collected after four weeks of culture on CIM medium. Callus were carefully isolated from the explants and transferred to a liquid medium for suspension culture. Collected callus can also keep on callus maintenance medium, which is same as CIM, for maintenance/multiplication first and then go suspension culture when there is enough callus for suspension culture.

Cell suspension culture. About 1 gram of callus is transferred to a 125 ml flask which contains 15 ml rose cell suspension culture medium (RSM). All flasks are sealed with double layer heavy duty autoclaved aluminum foil. Suspension cultures are kept in a cell culture incubator in the dark with a preset temperature of 28±2° C., the shaker rotates at the speed of 110 rpm. The RSM is refreshed every 7 days after the culture initiation. The medium is refreshed by removing half (7.5-8.0 ml) of the old medium with a pipette and adding the same amount of fresh medium. Refreshment serves to remove toxic substances that are released by dead or degenerated cells and/or prevent excess change in medium osmolarity, normally resulting from the breakdown of sucrose by enzymes of dividing cells. During this culture period, close monitoring of the culture should be practiced. Somatic embryo competent callus with yellow color appears approximately 4 months after suspension culture initiation (FIG. 1B). The cell will multiplicate quickly and once the embryoids reach the size or 2-3 mm in diameter after 6 months of suspension culture, they are transferred to somatic embryo regeneration medium (ERM) (Table 2) to allow somatic embryo formation.

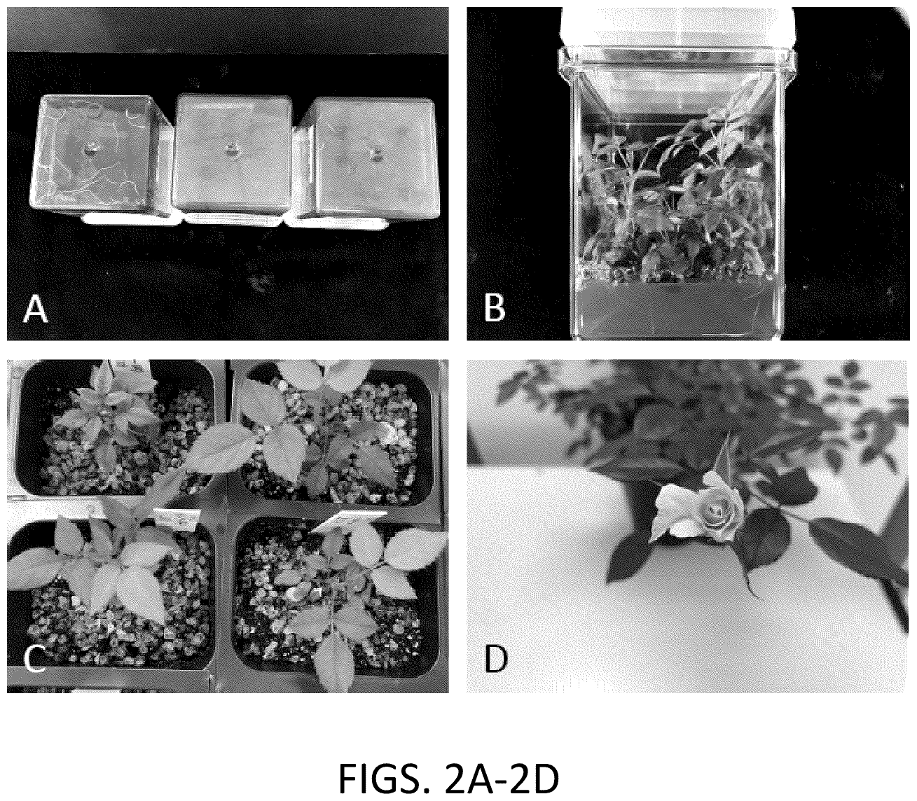

Somatic embryo induction. Embryoids or regenerable calluses, which are yellowish and compact (FIG. 1C) are transferred directly to ERM-I for somatic embryo regeneration/development. The emerged somatic embryos (FIGS. 1D-1F) are transferred to RIM for rooting (FIG. 2A). Somatic embryos of poor quality (no strong root development, or embryo clusters) can be first transferred to medium ERM-II (Table 2) to induce somatic embryo germination. Following such transfer. Somatic embryos are kept in the light at 25° C. for somatic embryo germination and development. Petri dishes with somatic embryos or regenerable calli on ERM-I are kept in the dark for four weeks. Once shoots grow to approximately 2-3 cm in height, they are transferred to magenta boxes containing root induction medium RIM (Table 2), for root initiation. About 28 days following the transfer to root induction medium RIM, rooted plantlets (FIG. 2B) are ready for transfer to a greenhouse or growth chamber for further growth. It is important to note that one regenerable callus or embryoid often gives rise to multiple somatic embryos/green plants.

Raising somatic embryo plants in a greenhouse. Once the root system is well established and the shoot reaches 5-6 cm or higher, plantlets can be transplanted directly to 32 cell trays. A tool (preferably a small spatula) is first employed to loosen the contact between the agar and the tube wall. The plantlet is then gently removed from the box and the agar is carefully rubbed off the roots. Care should be taken to avoid any unnecessary damage to the root system. In preparation for transplanting, pots are filled with soil and watered to saturation. Soils are premixed with fertilizers (N, P, K). The plantlets are then planted in pots in such a way that roots are placed in a hole and soil is gently pressed around the plant by fingers to enable good root-soil contact. The tray is covered by a dome to keep moisture. Plant trays are watered once a day or whenever the soil appears to have dried somewhat. It is beneficial to keep a high humidity for the first few days following transplanting. About 10 to 15 days after transplanting, plantlets are established in the cells and begin to grow rapidly (FIG. 2C). In a month (varies with the maturity date of genotypes), flower buds emerge from plants (FIG. 2D), regenerated plants show normal flower color and plant shape with the mother plant.

TABLE 2
Media formulations for rose cell suspension culture and regeneration.

| | CIM | RSM | ERM-I | ERM-II | RIM |
|---|---|---|---|---|---|
| | medium | medium | medium | medium | medium |
| Media components | (mg/L) | (mg/L) | (mg/L) | (mg/L) | (mg/L) |
| Macro salts | | | | | |
| NH₄NO₃ | 50-1000 | 150-2500 | 150-2000 | 150-2000 | 150-2000 |
| KNO₃ | 50-1000 | 150-2500 | 150-2000 | 150-2000 | 150-2000 |
| CaCl₂ (anhydrous) | 50-1000 | 150-2500 | 150-2000 | 150-2000 | 150-2000 |
| KH₂PO₄ | 50-1000 | 150-2500 | 150-2000 | 150-2000 | 150-2000 |
| MgSO₄•7H₂O | 50-1000 | 150-2500 | 150-2000 | 150-2000 | 150-2000 |
| Iron source | | | | | |
| FeSO₄•7H₂O | 25-40 | 25-40 | 35-60 | 25-40 | 25-250 |
| Na₂EDTA | 25-40 | 25-40 | 35-60 | 25-40 | 25-250 |
| Sequestrene | | | | | 25-250 |
| Micro salts | | | | | |
| H₃BO₃ | 0.2-25 | 0.5-5.0 | 0.02-25 | 0.02-25 | 0.02-25 |
| KI | 0.2-25 | 0.5-5.0 | 0.02-25 | 0.02-25 | 0.02-25 |
| MnSO₄•4H₂O | 0.2-25 | 0.5-5.0 | 0.02-25 | 0.02-25 | 0.02-25 |
| ZnSO₄•7H₂O | 0.2-25 | 0.5-5.0 | 0.02-25 | 0.02-25 | 0.02-25 |
| CoCl₂•6H₂O | 0.2-25 | — | 0.02-25 | 0.02-25 | 0.02-25 |
| CuSO₄•5H₂O | 0.2-25 | — | 0.02-25 | 0.02-25 | 0.02-25 |
| Na₂MoO4•2H₂O | 0.2-25 | — | 0.02-25 | 0.02-25 | 0.02-25 |
| Vitamins | | | | | |
| Myo-Inositol | 0.1-100 | — | 0.01-150 | 0.05-150 | 0.05-150 |
| Nicotinic acid | 0.1-100 | 0.1-250 | 0.01-150 | 0.05-150 | 0.05-150 |
| Pyridoxine | 0.1-100 | — | 0.01-150 | 0.05-150 | 0.05-150 |
| Thiamine HCl | 0.1-100 | 0.1-250 | 0.01-150 | 0.05-150 | 0.05-150 |
| Glycine | 0.1-100 | 0.1-250 | 0.01-150 | 0.05-150 | 0.05-150 |
| L-Proline | 0.1-100 | 0.1-250 | 0.01-150 | 0.05-150 | 0.05-150 |
| Asparagine | — | 0.1-250 | 0.01-150 | — | — |
| Glutamine | — | 0.1-250 | 0.01-150 | — | — |
| Boitin | — | — | 0.01-150 | — | — |
| Growth regulators | | | | | |
| BA | — | — | — | 0.1-2.5 | — |
| KT | — | 0.1-1.5 | — | — | — |
| TDZ | — | — | — | — | — |
| 2,4-D | 1.0-5.0 | 0.1-1.5 | 0.1-1.0 | — | — |
| NAA | 1.0-5.0 | — | — | 0.1-2.5 | — |
| IBA | — | — | — | — | 0.1-2.5 |
| IAA | — | — | — | — | 0.1-2.5 |
| Other components | | | | | |
| Sucrose | 10-20 | 25-75 | 25-50 | 25-50 | 25-50 |
| Maltose | — | 50-700 | — | — | — |
| Gelrite | 2-6 | — | — | — | — |
| Agar | — | — | 5-10 | 5-10 | 5-10 |
| PH | 5.0-6.5 | 5.0-6.5 | 5.0-6.5 | 5.0-6.5 | 5.0-6.5 |

1. A method of producing a rose plant using somatic embryogenesis, the method comprising:
culturing at least one rose plantlet leaf in callus induction medium (CIM);
isolating at least one callus from the at least one rose plantlet leaf and transferring the at least one callus into rose cell suspension culture media (RSM);
isolating at least one embryoid produced from the at least one callus and transferring the at least one embryoid into a somatic embryo regeneration medium (ERM-I); and
isolating at least one shoot produced from the at least one embryoid and transferring the at least one shoot to root induction media (RIM);
wherein the at least one shoot develops into a regenerated rose plant.

2. The method of claim 1, wherein the method further comprises isolating an embryoid produced from the callus and transferring the embryoid into a somatic embryo regeneration medium II (ERM-II), wherein the embryoid has insufficient root development and/or embryo clustering.

3. The method of claim 1, wherein the at least one rose plantlet leaf is from an in vitro plantlet.

4. The method of claim 1, wherein the at least one rose plantlet leaf is cultured with its abaxial side in contact with the CIM.

5. The method of claim 1, wherein the at least one callus is collected about 4 weeks after being cultured in the CIM.

6-7. (canceled)

8. The method of claim 1, wherein the RSM is refreshed about every 7 days after culture initiation.

9. The method of claim 1, wherein the at least one callus is cultured in RSM for at least about 4 months, or until the at least one callus becomes an embryoid that is at least 2 mm in diameter.

10. (canceled)

11. The method of claim 1, wherein the at least one embryoid is cultured in ERM-I for at least about 4 weeks, or until the at least one embryoid becomes a shoot that is at least 2 cm in height.

12-24. (canceled)

25. A rose cell suspension culture media (RSM) comprising:
at least one macro salt;
at least one iron source;
at least one micro salt;
at least one vitamin; and
at least one growth regulator;
wherein culturing at least one rose callus in RSM is sufficient to produce at least one embryoid.

26. The media of claim 25, wherein the at least one macro salt is present at a concentration ranging from about 150 mg/L to about 2500 mg/L.

27. The media of claim 25, wherein the at least one macro salt is selected from the group consisting of NH₄NO₃, KNO₃, CaCl₂, KH₂PO₄, and MgSO₄.

28. The media of claim 25, wherein the at least one iron source is present at a concentration ranging from about 25 mg/L to about 40 mg/L.

29. The media of claim 25, wherein the at least one iron source is selected from the group consisting of FeSO₄ and Na₂EDTA.

30. The media of claim 25, wherein the at least one micro salt is present at a concentration ranging from about 0.5 mg/L to about 5.0 mg/L.

31. The media of claim 25, wherein the at least one micro salt is selected from the group consisting of H₃BO₃, KI, MnSO₄, and ZnSO₄.

32. The media of claim 25, wherein the at least one vitamin is present at a concentration ranging from about 0.1 mg/L to about 250 mg/L.

33. The media of claim 25, wherein the at least one vitamin is selected from the group consisting of Nicotinic acid, Thiamine HCl, Glycine, L-Proline, Asparagine, and Glutamine.

34. The media of claim 25, wherein the at least one growth regulator is present at a concentration ranging from about 0.1 mg/L to about 1.5 mg/L.

35. The media of claim 25, wherein the at least one growth regulator is selected from the group consisting of Kinetin (KT) and 2,4-Dichlorophenoxyacetic acid (2,4-D).

36. The media of claim 25, wherein the media further comprises at least one of sucrose and/or maltose, and/or wherein the media comprises a pH ranging from about 5.0 to about 6.5.

37-76. (canceled)
